(19) United States
(12) Patent Application Publication
(10) Pub. No.: US 2025/0128022 A1
(43) Pub. Date: Apr. 24, 2025

(54) CATHETER WITH TRANSITIONAL SEGMENT IN WOVEN LAYER FOR ATTACHED TUBE

(71) Applicant: Boston Scientific Medical Device Limited, Galway (IE)

(72) Inventors: Miles Montgomery, Holland Landing (CA); Ros Thomas Brannick, Picton (CA); Luke Keaveney, Georgetown (CA)

(21) Appl. No.: 18/918,958

(22) Filed: Oct. 17, 2024

Related U.S. Application Data
(60) Provisional application No. 63/591,719, filed on Oct. 19, 2023.

(51) Int. Cl. A61M 25/00

(52) U.S. Cl. CPC A61M 25/0054; A61M 25/005; A61M 2207/10

(57) ABSTRACT

A catheter comprises a shaft defining a lumen and having a distal section. The shaft includes an inner member and a support member disposed on the inner member. The support member includes a proximal woven portion and a distal woven portion separated by an intermediate nonwoven portion. The distal woven portion includes an outer surface. The proximal woven portion is coupled to the distal woven portion by interconnecting threads of the intermediate portion. The shaft further includes an electrode on the distal section of the shaft, a tube extending along the shaft, and a lead conductor disposed within the tube and electrically coupled to the electrode. The tube extends radially underneath the proximal woven portion, extends on the outer surface along the distal woven portion, and extends from underneath the proximal woven portion to the outer surface at the intermediate nonwoven portion through the interconnecting threads.

### CROSS REFERENCE TO RELATED APPLICATIONS

This application claims priority to U.S. Provisional Patent Application No. 63/591,719 entitled, “CATHETER WITH TRANSITIONAL SEGMENT IN WOVEN LAYER FOR ATTACHED TUBE,” filed Oct. 19, 2023, which is hereby incorporated by reference in its entirety.

### TECHNICAL FIELD

The present disclosure relates to medical devices for catheterization procedures, such as medical devices for electrophysiological procedures. More specifically, the present disclosure relates to catheters, catheter systems, and methods for manufacturing catheters.

### BACKGROUND

Various medical fields use different types of catheters to achieve access to a physiological site in medical procedures. For instance, electrophysiological procedures involve guiding catheters into the heart and tracking the location of the catheters with respect to the heart. Catheter ablation is minimally invasive electrophysiological procedure to treat a variety of heart conditions such as supraventricular and ventricular arrhythmia. Example catheters used in catheter ablation can include mapping catheters, ablation catheters, guiding sheaths, dilators, and other medical tools, which can be referred to as catheters in this disclosure. Electrophysiological procedures can involve the visualization of the heart, heart activity, and the position of the catheters within the heart. A common visualization system involves the use of fluoroscopy, which can expose the patient and clinician to radiation. Electroanatomical mapping is an alternative visualization technique that does not involve the use of radiation.

Electroanatomical mapping allows a clinician to accurately determine the location of an arrhythmia, define cardiac geometry in three dimensions, delineate areas of anatomic interest, and permits imaging of the catheters for positioning and manipulation. Catheters used with electroanatomical mapping systems can include tracking capabilities, such as navigation enabled or impedance-based tracking methodologies. Navigation-enabled catheters use magnetic sensors in the presence of magnetic fields to track the location and orientation of the catheters. But not all catheters include magnetic sensors. Impedance-based catheters use electrodes in the presence of electric fields to track the catheters.

### SUMMARY

In an Example 1, a catheter, comprising: an elongated shaft defining a lumen along a longitudinal axis and having a distal section, the shaft comprising: a longitudinally extending inner member defining the lumen, and a support member disposed on the longitudinally extending inner member, the support member including a proximal woven portion and a distal woven portion separated by an intermediate nonwoven portion, the distal woven portion having an outer surface, the proximal woven portion coupled to the distal woven portion by a plurality of longitudinally extending interconnecting threads of the intermediate portion; an electrode disposed on the distal section of the elongated shaft; an elongated tube extending longitudinally along the elongated shaft; and a lead conductor disposed within the tube and electrically coupled to the electrode; wherein the tube extends longitudinally along the proximal woven portion radially underneath the proximal woven portion, extends longitudinally on the outer surface along the distal woven portion, and extends from underneath the proximal woven portion to the outer surface at the intermediate nonwoven portion through the interconnecting threads.

In an Example 2, the catheter of Example 1, wherein the proximal woven portion and the distal woven portion includes a stainless-steel braided material.

In an Example 3, the catheter of Example 2, wherein the braided material includes conductive threads.

In an Example 4, the catheter of any of Examples 1-3, wherein the elongated shaft includes an outer layer disposed on the support member.

In an Example 5, the catheter of Example 4, wherein the outer layer includes a polyether block amide.

In an Example 6, the catheter of any of Examples 1-5, wherein the electrode is ring electrode.

In an Example 7, the catheter of any of Examples 1-6, wherein the electrode includes a plurality of electrodes, and the tube includes a plurality of tubes.

In an Example 8, the catheter of any of Examples 1-7, wherein the shaft includes a proximal end, the proximal end coupled to an electrical connector, and the lead conductor electrically coupled to the electrical conductor.

In an Example 9, the catheter of any of Examples 1-8, wherein the proximal braided member includes an inner surface, and the tube extends longitudinally along the inner surface.

In an Example 10, the catheter of any of Examples 1-9, wherein the catheter is a guide catheter having a proximal end coupled to a handle.

In an Example 11, the catheter of any of Examples 1-10, wherein the proximal woven portion includes a first set of warp and weft threads, the distal woven portion includes a second set of warp and weft threads, and warp threads of the first set extend to the warp threads of the second set as the interconnected threads.

In an Example 12, the catheter of any of Examples 1-11, wherein the proximal woven portion and the distal woven portion are characterized by a picks per inch of greater than zero, and the intermediate nonwoven portion is characterized by a picks per inch of zero.

In an Example 13, the catheter of any of Examples 1-12, wherein the catheter includes one of a dilator and a guide catheter.

In an Example 14, the catheter of any of Examples 1-12, wherein the shaft includes a distal tip section having an ablation electrode assembly.

In an Example 15, the catheter of any of Examples 1-14, wherein the tube is attached to the outer surface along the distal woven portion.

In an Example 16, a catheter, comprising: an elongated shaft defining a lumen along a longitudinal axis and having a distal section, the shaft comprising: a longitudinally extending inner member defining the lumen; and a support member disposed on the longitudinally extending inner member, the support member including a proximal woven portion and a distal woven portion separated by an intermediate nonwoven portion, the distal woven portion having an outer surface, the proximal woven portion coupled to the distal woven portion by a plurality of longitudinally extending interconnecting threads of the intermediate portion; an electrode disposed on the distal section of the elongated shaft; an elongated tube extending longitudinally along the elongated shaft; and a lead conductor disposed within the tube and electrically coupled to the electrode; wherein the tube extends longitudinally along the proximal woven portion radially underneath the proximal woven portion, extends longitudinally on the outer surface along the distal woven portion, and extends from underneath the proximal woven portion to the outer surface at the intermediate nonwoven portion through the interconnecting threads.

In an Example 17, the catheter of Example 16, wherein the proximal woven portion and the distal woven portion includes a stainless-steel braided material.

In an Example 18, the catheter of Example 17, wherein the braided material includes conductive threads.

In an Example 19, the catheter of Example 16, wherein the elongated shaft includes an outer layer disposed on the support member.

In an Example 20, the catheter of Example 19, wherein the outer layer includes a polyether block amide.

In an Example 21, the catheter of Example 16, wherein the electrode is ring electrode.

In an Example 22, the catheter of Example 16, wherein the electrode includes a plurality of electrodes, and the tube includes a plurality of tubes.

In an Example 23, the catheter of Example 16, wherein the shaft includes a proximal end, the proximal end coupled to an electrical connector, and the lead conductor electrically coupled to the electrical conductor.

In an Example 24, the catheter of Example 16, wherein the proximal braided member includes an inner surface, and the tube extends longitudinally along the inner surface.

In an Example 25, the catheter of Example 16, wherein the catheter is a guide catheter having a proximal end coupled to a handle.

In an Example 26, the catheter of Example 16, wherein the proximal woven portion includes a first set of warp and weft threads, the distal woven portion includes a second set of warp and weft threads, and warp threads of the first set extend to the warp threads of the second set as the interconnected threads.

In an Example 27, the catheter of Example 16, wherein the proximal woven portion and the distal woven portion are characterized by a picks per inch of greater than zero, and the intermediate nonwoven portion is characterized by a picks per inch of zero.

In an Example 28, the catheter of Example 16, wherein the catheter includes one of a dilator and a guide catheter.

In an Example 29, the catheter of Example 16, wherein the shaft includes a distal tip section having an ablation electrode assembly.

In an Example 30, the catheter of Example 16, wherein the tube is attached to the outer surface along the distal woven portion.

In an Example 31, a system for tracking a catheter during an electrophysiology procedure on a patient, the system comprising: a catheter disposable within a patient, the catheter comprising: an elongated shaft defining a lumen along a longitudinal axis and having a distal section, the shaft comprising: a longitudinally extending inner member defining the lumen; and a support member disposed on the longitudinally extending inner member, the support member including a proximal woven portion and a distal woven portion separated by an intermediate nonwoven portion, the distal woven portion having an outer surface, the proximal woven portion coupled to the distal woven portion by a plurality of longitudinally extending interconnecting threads of the intermediate portion; a tracking electrode disposed on the distal section of the elongated shaft; an elongated tube extending longitudinally along the elongated shaft; and a lead conductor disposed within the tube and electrically coupled to the electrode; wherein the tube extends longitudinally along the proximal woven portion radially underneath the proximal woven portion, extends longitudinally on the outer surface along the distal woven portion, and extends from underneath the proximal woven portion to the outer surface at the intermediate nonwoven portion through the interconnecting threads; and a controller operably coupled to the tracking electrode, the controller configured to receive an electrical signal from the tracking electrode and determine a position of the catheter with respect to the patient.

In an Example 32, the system of Example 31, wherein the controller if further configured to generate an electroanatomical map of the heart of the patient.

In an Example 33, the system of Example 31, wherein the controller is configured to determine the position of the catheter via an impedance-based process.

In an Example 34, a method of manufacturing a catheter, the method comprising: forming a distal section of an elongate shaft from an inner member defining a lumen, a support member disposed on the inner member, the support member including a proximal woven portion, and a distal woven portion separated by an intermediate nonwoven portion, the distal woven portion having an outer surface, the proximal woven portion coupled to the distal woven portion by a plurality of longitudinally extending interconnecting threads of the intermediate portion; attaching an elongated tube longitudinally along the elongated shaft, wherein the tube extends longitudinally along the proximal woven portion radially underneath the proximal woven portion, extends longitudinally on the outer surface along the distal woven portion, and extends from underneath the proximal woven portion to the outer surface at the intermediate nonwoven portion through the interconnecting threads; attaching an electrode on the distal section of the elongated shaft; and electrically coupling a lead conductor disposed in the tube to the electrode.

In an Example 35, the method of Example 34, wherein the forming of the distal section includes forming one of the proximal woven portion and the distal woven portion by weaving threads, forming the intermediate portion by longitudinally pulling the threads and not weaving the threads, and forming the other of the proximal woven portion and the distal woven portion by weaving the threads.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, the example clinical setting having an example electrophysiology system.

FIG. 2 is a schematic diagram illustrating an example catheter that can be used with the example electrophysiology system. of FIG. 1.

FIG. 3A is a schematic diagram illustrating from a side view an example feature of the example catheter of FIG. 2.

FIG. 3B is a schematic diagram illustrating from a sectioned view the example feature of the example catheter of FIG. 3A.

FIG. 4 is a block diagram illustrating an example method of manufacturing the catheter of FIG. 2.

FIG. 5 is a block diagram illustrating an example method of a feature of the method of manufacturing the catheter of FIG. 4.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) of the features in an example used across all examples. Thus, no one figure should be interpreted as having any dependency or requirement related to any single component or combination of components illustrated therein. Additionally, various components depicted in a figure may be, in examples, integrated with various ones of the other components depicted therein (or components not illustrated), all of which are within the ambit of the present disclosure.

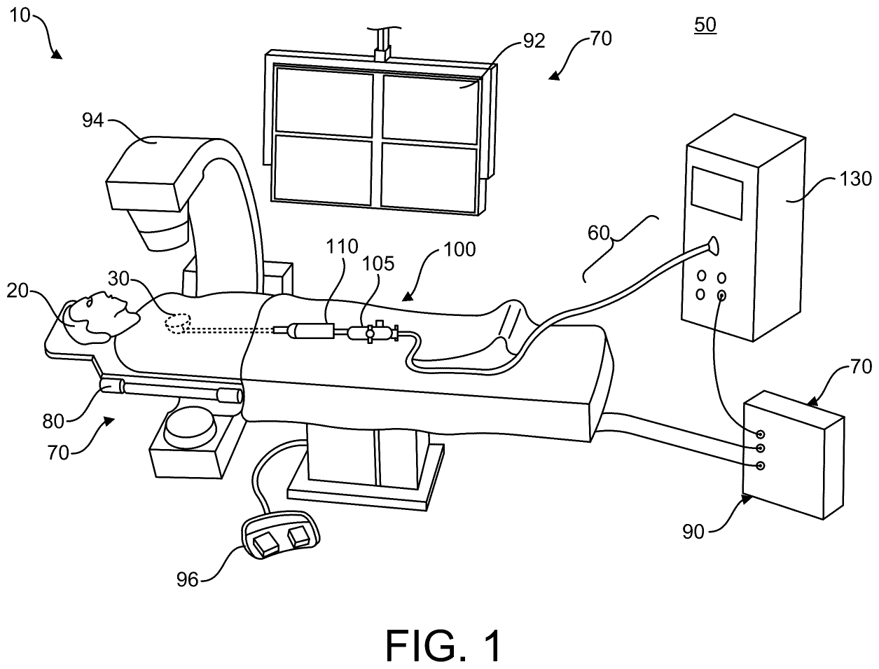

FIG. 1 illustrates an example clinical setting 10 for treating a patient 20, such as for treating a heart 30 of the patient 20, using an electrophysiology system 50, in accordance with the disclosure. The electrophysiology system 50 includes a catheter system 60 and an electroanatomical mapping (EAM) system 70. The example catheter system 60 includes an elongated catheter assembly 100, which includes an ablation catheter 105 and a catheter sheath 110, and an electroporation console 130. Additionally, the catheter system 60 includes various connecting elements, such as cables, that operably connect the components of the catheter system 60 to one another and to the components of the EAM system 70. In general, the EAM system 70 includes a localization field generator 80, a mapping and navigation controller 90, and a display 92. Also, the clinical setting 10 can include additional equipment such as imaging equipment 94 (represented by the C-arm) and various controller elements, such as a foot controller 96, configured to allow an operator to control various aspects of the electrophysiology system 50. The clinical setting 10 may have other components and arrangements of components that are not shown in FIG. 1.

The sheath 110 is operable to provide a delivery conduit through which the catheter 105 can be deployed to the specific target sites within the patient's heart 30. Access to the patient's heart can be obtained through a vessel, such as a peripheral artery or vein. Once access to the vessel is obtained, the catheter 105 can be navigated to within the patient's heart, such as within a chamber of the heart.

The example catheter system 60 is configured to deliver ablation energy to targeted tissue in the patient's heart 30 to create cell death in tissue, for example, rendering the tissue incapable of conducting electrical signals. An elongated catheter assembly, such as catheter assembly 100, can include a plurality of coaxially disposed catheters. For instance, a catheter defines a longitudinal axis that passes through a centroid of a cross section of the catheter, such as the centroid of a cross section of a shaft of catheter 105 or a centroid of a cross section of a main lumen of a sheath 110. As shown, the catheter 105 is disposed within the sheath 110. The catheters 105, 110 are movable with respect to each other along the longitudinal axis.

The example catheter 105 includes an elongated catheter shaft and distal end configured to be deployed proximate to the target tissue, such as within a chamber of the patient's heart. The distal end may include a basket, balloon, spline, configured tip, or other deployment mechanism to effect treatment. The deployment mechanism can include an electrode assembly or array having a plurality of ablation electrodes. Each of the plurality of ablation electrodes is electrically coupled to a corresponding elongated lead conductor that extends along the shaft to a catheter proximal end. The lead conductors can be electrically coupled to a plug in the proximal region of the catheter 105, such as a plug configured to be mechanically and electrically coupled to the console 130, for example, either directly or via intermediary electrical conductors such as cabling. In one example, the console 130 is configured to provide an electrical signal, such as a plurality of concurrent or space-apart-time electrical signals, to the electrically connected catheter 105 along lead conductors to the spaced-apart electrodes to effect ablation.

The console 130 is configured to control aspects of the catheter system 60. The console 130 includes a controller, such one or more controllers, processors, or computers, that executes instructions or code, such as processor-executable instructions, out of a non-transitory computer readable medium, such as a memory device, or memory, to cause, such as control or perform, the aspects of the electroporation catheter system 60. The memory can be part of the one or more controllers, processors, or computers, or part of memory device accessible through a computer network. Examples of computer networks include a local area network, a wide area network, and the internet.

The EAM system 70 can be operable to track the location of the various components of the catheter system 60, and to generate high-fidelity three-dimensional anatomical and electro-anatomical maps of the heart, including portions of the heart such as cardiac chambers of interest or other structures of interest such as the sinoatrial node or atrioventricular node. In one illustrative example, the EAM system 70 can include the RHYTHMIA™ HDx mapping system marketed by Boston Scientific Corporation. The mapping and navigation controller 90 of the EAM system 70 includes one or more controllers, such as microprocessors or computers, that execute code out of memory to control or perform functional aspects of the EAM system 70, in which the memory, can be part of the one or more controllers, microprocessors, computers, or part of a memory device accessible through a computer network.

The EAM system 70 can generate a localization field, via the magnetic field generator 80, to define a localization volume about the heart 30, and a location sensor or sensing element on a tracked device, such as sensors on the electroporation catheter 105, generate an output that can be processed by the mapping and navigation controller 90 to track the location and orientation of the sensor or sensors, and consequently, the corresponding device, within the localization volume. In the illustrated example, the device tracking is accomplished using magnetic tracking techniques, in which the field generator 80 is a magnetic field generator that generates a magnetic field defining the localization volume, and location sensors on the tracked devices are magnetic field sensors.

In other examples, impedance tracking methodologies may be employed to track the locations of the various devices. In such examples, the localization field is a set of independently oriented and spatially varying electric fields generated, for example, by an external field generator arrangement, such as surface electrodes, by intra-body or intra-cardiac devices, such as an intracardiac catheter, or both. In these examples, the location sensing elements can constitute tracking electrodes on the tracked catheters that generate outputs received and processed by the mapping and navigation controller 90 to track the location of the various location sensing electrodes within the localization volume. For instance, an impedance tracking methodology can employ the use of a patch electrode (not shown) attached to the patient's body, a current or impedance based can be determined between the tracking electrode on the catheter and the patch electrode.

The EAM system 70 can be equipped for magnetic tracking capabilities, impedance tracking capabilities, or for both magnetic and impedance tracking capabilities. Regardless of the tracking methodology employed, the EAM system 70 utilizes the location information for the various tracked devices, along with cardiac electrical activity acquired by, for example, the electroporation catheter 105 or another catheter or probe equipped with sensing electrodes, to generate, and display via the display 92, detailed three-dimensional geometric anatomical maps or representations of the heart tissue and voids such as cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map or the electro-anatomical map.

In the case of impedance-based tracking with the EAM system 70, the catheters include tracking electrodes disposed on the deflectable portions of the catheter shafts. Multiple tracking electrodes can be employed on the deflectable portions of the catheters for the EAM system 70 to detect and recreate the curvature of the catheters in the body. In one example, each tracking electrode is coupled to a corresponding lead conductor, or lead wire, which extends along the shaft to the proximal portion where it is coupled to an electrical connector. The electrical connector can be coupled to the EAM system 70 such as via cables.

Several constraints are employed in the design and implementation of tracking electrodes and associated lead conductors. Among these constraints include that each tracking electrode and associated lead conductor are to be electrically isolated from one another as well as from other conductive material in the catheter such as a conductive braided member along the length of the shaft. Additionally, precise placement of the tracking electrode on the shaft is desired. For instance, the tracking electrode can be radiopaque, and clinicians can use the tracking electrode to visualize placement of the catheter while using fluoroscopy. Also, electrode location with respect to the catheter elements and interelectrode spacing are programmed parameters in several tracking and mapping software programs, and three-dimensional reconstruction and modeling is performed with electrode spacing as a constraint in the modeling curve. Thus, the design and implementation of catheters using tracking electrodes can benefit from ready access to lumens carrying conductor leads.

The disclosure relates to a catheter having a segment in the braided member that enables a tube carrying a lead conductor to transfer from underneath a braided member to the outer surface of the braided member. The segment can provide for shaft stiffness, pushability, kink resistance, flexibility, and torque transmission, which can be undesirably affected by removing large portions of the braided member to pass the tube. The catheter includes an elongated shaft defining a lumen along a longitudinal axis and having a distal segment. The elongated shaft includes a longitudinally extending inner member defining the lumen and a support member disposed on the longitudinally extending inner member. The support member includes a proximal woven portion and a distal woven portion separated by an intermediate nonwoven portion. The distal woven portion includes an outer surface. The proximal woven portion is coupled to the distal woven portion by a plurality of longitudinally extending interconnecting threads of the intermediate portion. An electrode is disposed on the distal portion of the elongated shaft. An elongated tube extends longitudinally along the elongated shaft. A lead conductor is disposed within the tube and electrically coupled to the electrode. The tube extends longitudinally along the proximal woven portion radially underneath the proximal woven portion, extends longitudinally on the outer surface along the distal woven portion, and extends from underneath the proximal woven portion to the outer surface at the intermediate nonwoven portion through the interconnecting threads.

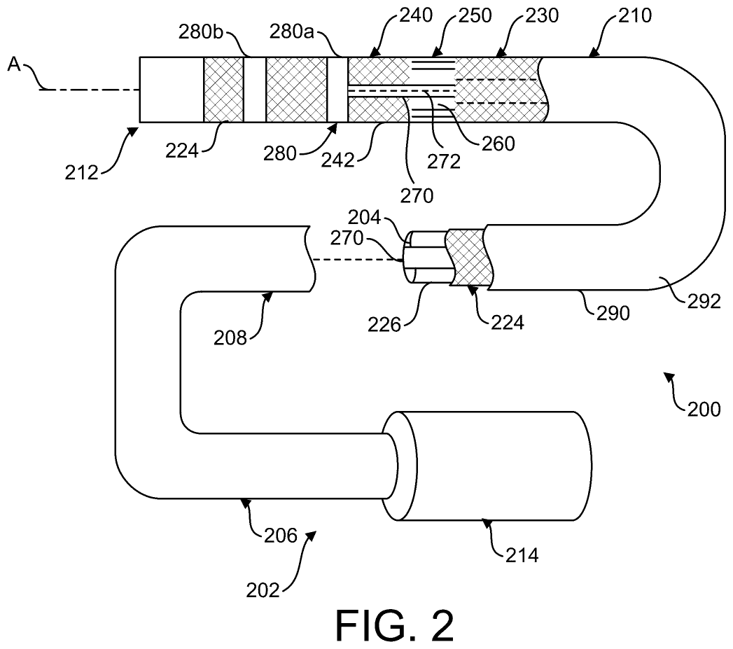

FIG. 2 illustrates an example catheter 200 that can be used in with catheter assembly 100. For instance, catheter 200 can be further configured as a guide catheter, dilator, ablation catheter or other flexible, deflectable medical tool that can be tracked via an impedance tracking system such as EAM system 70. The catheter 200 includes an elongated shaft 202, such as an elongated and flexible shaft 202 defining a longitudinal axis A. The shaft 202 also defines a main lumen 204 along the longitudinal axis A and has a proximal section 206, a longitudinal section 208, and a distal section 210. The distal section 210 includes a distal tip section 212. The proximal section 206 can be coupled to a handle 214 proximal to the shaft 202.

The shaft 202 includes a plurality of components disposed along the distal section 210 and defining a main lumen 204. The shaft 202 includes a support member 224 disposed between a longitudinally extending inner member 226 and a longitudinally extending outer layer 290. The inner member 226 defines the main lumen 204, and the support member 224 is disposed on the inner member 226. In various examples, the inner layer, the outer layer, or both are formed of multiple layers. The support member 224 includes an elongated and flexible proximal portion of the support member, or proximal support member 230 disposed on the inner member 226, and the support member 224 includes a distal portion of the support member, or distal support member 240 disposed on the inner member 226. The distal support member 240 includes an outer surface 242. The distal support member 240 is longitudinally spaced apart from the proximal support member 230 along the axis A at an intermediate portion 250 of the support member 224. The distal support member 240 is longitudinally spaced-apart from the proximal support member 230 at a suitable distance. In various embodiments, the range of the spaced apart distance can be 1 millimeter to 20 millimeters, for instance, the intermediate portion is in a range of 2 millimeters to 10 millimeters. In the illustrated example, the proximal and distal support members 230, 240 include a woven, or braided fabric, such as a stainless-steel braided fabric. For illustration, the proximal and distal support members 230, 240 are presented as proximal and distal braided members 230, 240.

The intermediate support member 250 is disposed between the proximal support member 230 and the distal support member 240. Whereas the proximal support member 230 and distal support member 240 are formed of woven, or braided fabric, such as from stainless steel threads, having a warp and a weft, the intermediate support member 250 is not a woven fabric. The intermediate support member 250 includes longitudinally extending threads that connect the proximal support member 230 and distal support member 240. The longitudinally extending interconnecting threads of the intermediate support member 250 can include warp or weft threads of the proximal support member 230 and the distal support member 240 and, in some embodiments, are integral to the fabric of the proximal support member 230 and the distal support member 240. In one example, a longitudinal weaving process is used to construct the support member 224, such as from the proximal end to the distal end. In this construction, the weaving process is paused to form the intermediate support member, and threads are dragged longitudinally from the proximal support member 230 and the distal support member 240, or vice versa, to form the intermediate support member 250. Whereas the proximal support member 230 and the distal support member 240 are characterized by a tight weave with miniscule interstitial spacing between the threads, the intermediate support member 250 include larger openings 260 between the longitudinally extending threads.

An elongated tube 270 extends longitudinally along the shaft 202 and carries a lead conductor 272 that is coupled to an electrode 280 (e.g., a tracking electrode) disposed on the distal portion 208 of the shaft 202. The tube 270 extends longitudinally along the shaft 202 radially underneath the proximal braided member 230 through the length of the proximal braided member 230. In various examples, the proximal braided member 230 includes an inner surface 232. The tube 270 extends longitudinally along the proximal braided member 230 radially underneath the inner surface 232 such as between the inner surface 232 and the inner member 226. The tube 270 extends from underneath the proximal braided member 230 to the outer surface 242 of the distal braided member 240 at the intermediate portion 250. To pass from underneath the proximal braided member 230 to the outer surface 242 of the distal braided member 240, the tube 270 can pass through the opening 260 in the intermediate member 250 between the longitudinally extending interconnecting threads of the intermediate member and extends longitudinally along an outer surface 242 of the distal braided member 240. The tube 270 can be attached to the outer surface 242 of the distal braided member 240.

According to various examples, the distal portion 210 of the shaft 202 includes a plurality of longitudinally spaced-apart tracking electrodes 280a . . . 280n, and each tracking electrode 280a . . . 280n can be electrically coupled to a respective lead conductor carried in a corresponding tube extending longitudinally along the shaft 202 and terminated at the proximal portion 206. The tracking electrodes 280 are illustrated as ring electrodes in the shaft. In certain examples, the tracking electrodes 280a . . . 280n are configured for use with an impedance-based tracking system. The proximal portion 206 or handle 214 can include an electrical connection that can be coupled to an impedance-based tracking system, such as EAM system 70. In one example, the electrical connection is available under the trade designation LEMO. In some examples, the shaft 202 further includes the outer layer 290 disposed over braided member 224 and tube 270 to form an outermost surface 292 of the shaft 202. The tracking electrodes 280a . . . 280n are exposed on the outermost surface 292 of the catheter 200.

The catheter 200 can include additional components for a selected implementation. In some examples, such as in the case of the catheter 200 configured as a guide sheath or dilator, the proximal portion 206 or handle 214 can include a hemostatic valve that can be coupled to a source of irrigation fluid and a port to receive catheters within the main lumen 204. In some examples, the distal tip section 212 can be configured as a dilator tip. In some examples the shaft 202 can include a liner layer (not shown) coaxial with the main lumen 204 to define an inner wall of the main lumen 204. In examples such as in which the catheter 200 is a guide sheath or dilator, the liner layer can be a thin wall constructed of polytetrafluoroethylene (PTFE). In some examples, such as in the case of the catheter 200 configured as an ablation catheter, the proximal portion 206 or handle can be coupled to a source of ablation energy, such as an electrical signal from console 130 or a cryogenic fluid. For instance, the main lumen 204 can be configured to carry electrical leads, such as leads to the ablation electrodes or other sensors, steering wires, or a conduit of irrigation fluid along the shaft 202 to the distal portion 210. In some examples, the distal tip section 212 can be configured to include an ablation electrode assembly or other sensors.

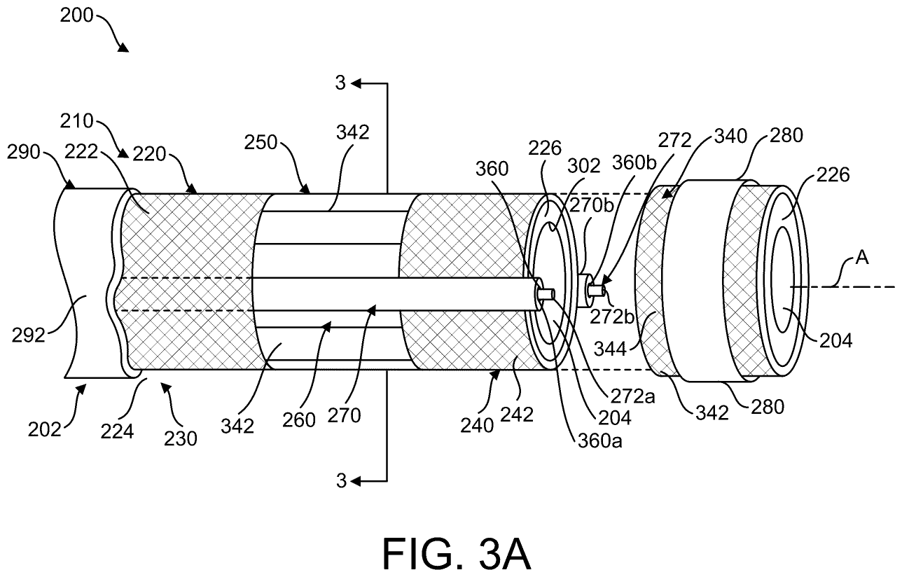

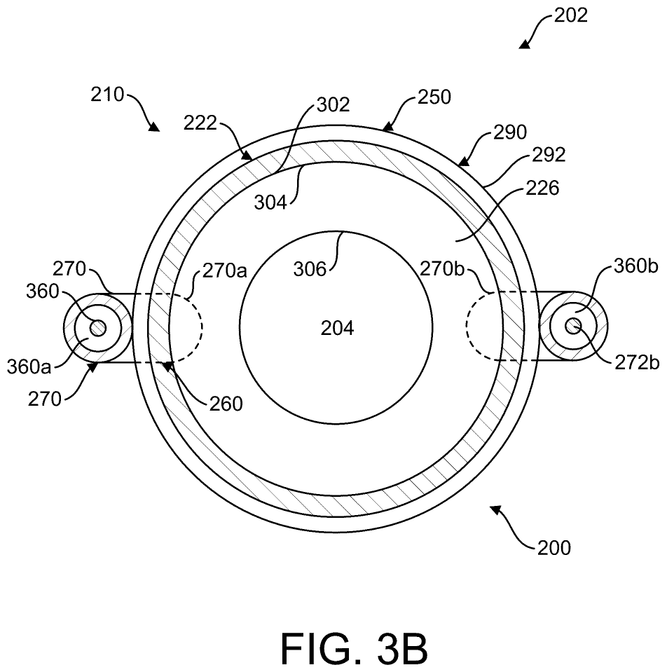

FIGS. 3A and 3B illustrate an example of catheter 200 having a reinforcement member as a tubular structure disposed in the breakout section of the support member between spaced-apart proximal and distal support members. FIG. 3A illustrates features of the catheter 200 from a perspective view, and FIG. 3B includes a cross section front view of the catheter 200, such as the distal portion 210, taken along line 3-3 of FIG. 3A. As shown, the support member 224 is constructed to include a braided fabric. In particular, the catheter 200 includes the elongated and flexible shaft 202. The shaft 202 includes the braided support member 224 and a longitudinally extending inner member 226, and the outer layer 290, which are coaxial with the main lumen 204. The longitudinally inner member 226 defines the main lumen 204. The support member 224 can include an inner side 306 disposed toward the main lumen 204 and opposite the outer side 222. The longitudinally extending inner member 226, such as a plurality of longitudinally extending inner layers, are disposed radially underneath the support member 224. The inner member 226 can include an outer side 304 (radially underneath the inner side 302 of the braided member 224) and an inner wall 306 defining the main lumen 204. In some examples, the shaft 202 can include a plurality of concentric or coaxially braided members along a longitudinal section of the shaft 202, such an innermost braided member and an outermost braided member. In certain examples, a single distal braided member 240 disposed along a longitudinal section of the shaft 202, i.e., a distal braided member 240 that is not concentric with another distal braided member, is also an outermost distal braided member such as the illustrated outermost distal braided member 240.

The support member 224 of the shaft 202 includes an elongated proximal portion, or proximal braided member 230, disposed on the inner member 226, and the support member 224 includes a distal portion, or distal braided member 240, disposed on the inner member 226. The distal braided member 240 includes an outer surface 242. The distal braided member 240 is longitudinally spaced apart from the proximal braided member 230 along the axis A as at an intermediate member 250 section of the support member 224. In some examples, the spacing of the intermediate member 250 between the proximal braided member 230 and the distal braided member 240 is in a range of 2 millimeters to 10 millimeters, such as 5 millimeters. The intermediate member 250 includes spaced-apart threads that connect the proximal support member 230 and the distal support member 240, and the spacing between the threads 342 forms opening 260. The proximal braided member 230 is terminated distally at a first braided member distal end 320. The distal braided member 240 is terminated proximally at a second braided member proximal end 322. The first braided member distal end 320 is longitudinally spaced apart from the second braided member proximal end 322. In some embodiments, the intermediate member 250 is formed by threads 342 woven into the proximal support member 230 and the distal support member 240, such as warp or weft threads. coupled to the proximal braided member 230 and the distal braided member 240. For example, the first braided member distal end 320 is coupled to the second braided member proximal end 322 via the longitudinally extending interconnected threads 342 of the intermediate support member 250.

The support member 224, and in particular the proximal and distal braided members 230, 240, provides characteristics to the catheter 200 such as reduced kinking, wrinkling, or buckling of the shaft 202 and can provide enhanced balance for pushability, deflectability, and torque transmission such as during rotation about the longitudinal axis A. The braided members 230, 240 can be constructed from a woven fabric, such as woven fabric 340 or layer of braided threads, or strands or fibers, 342 that may form interstitial spaces 344 between the threads 342. In general, the interstitial spaces 344 between the threads 342 are too small to fit the tube 270 through. The braided members 230, 240 can further be characterized by the warp and weft and bias of the threads 342 as well as picks per inch. For instance, in some examples, the picks per inch can remain generally uniform along the entire longitudinal length of one or both of the braided members 230, 240. In some examples, the picks per inch can vary along portions of the longitudinal length of one or both of the braided members 230, 240. The braided members 230, 240 can be constructed from threads 342 that include stainless steel fibers, such as conductive fibers, or high strength polymer fibers, or as layers of different materials. The braided members 230, 240 can be constructed from the same type or of different types of woven fabric 340. The intermediate member 250, in various embodiments, includes an extension of threads 342 in a nonwoven form from the proximal braided member 230 to the distal braided member 240 drawn longitudinally across the inner member 226 rather than woven into a braided fabric. In one embodiment, the intermediate member 250 includes the threads 342 used in making the woven fabric 340 of the proximal and distal braided member 230, 240. In contrast to the proximal and distal braided member 230, 240, the threads 342 can be manipulated so as to form the opening 260 as a spacing between the threads 342 large enough to fit the tube 270 through.

The elongated tube 270 extends longitudinally underneath the proximal braided member 230. In one example, the elongate tube 270 contacts the inner surface 232 of the proximal braided member 230 as the tube 270 traverses the proximal braided member 230. The elongated tube 270 contact an outer surface 242 of the distal braided member 240. For example, the elongated tube 270 is abutted against the outer surface 242 of the distal braided member 240 or is glued (e.g., using an adhesive) or otherwise attached to the outer surface 242 of the distal braided member 240. For instance, a longitudinally extending coiled thread, such as a nylon thread or twine, can be wrapped around the braided member 224 and tube 270 to hold the tube 270 against at least the distal braided member 240. The tube 270 extends from within the intermediate member 250 through an opening 260 between the longitudinally extending threads 342 to the outer surface 242 of the distal braided member 240. In one example, the elongated tube 270 extends longitudinally and generally straight along the proximal braided member 230 and the distal braided member 240. The elongated tube 270 is configured from a suitable material, such as a polymer, and defines a longitudinally extending lead lumen 360 along the length of the tube 270. The tube 270 is configured to carry a lead conductor, such as lead wire 272, within the lead lumen 360. The lead wire 272 is configured to be electrically coupled to a tracking electrode 280 and carry an appropriate electrical signal from the electrical connector at the proximal portion 204 to the tracking electrode 280. In one example, the elongate tube 270 is of a substantially smaller diameter than the shaft 202, and the main lumen 204 has a substantially larger diameter than the diameter of the lead lumen 360.

In an embodiment of a catheter 200 including multiple tracking electrodes, an elongated tube carrying a lead wire is included for each tracking electrode. Each of the tubes extend longitudinally along the proximal braided member 230 radially underneath the proximal braided member 230 and longitudinally on the outer surface 242 along the distal braided member 240 and extends to the outer surface 242 through the intermediate support member 250. In one example, each elongate tube 270 extends through a separate opening 260 between different spaced-apart threads. In some examples, the catheter 200 can include two tracking electrodes and includes a first elongated tube 270a carrying a first lead wire 272a within first lead lumen 360a and a second elongated tube 270b carrying a second lead wire 272b within first lead lumen 360b. Multiple elongated tubes 270 can be radially spaced-apart radially underneath the proximal braided member 230 and on the outer side 242 of the distal braided member 240. The intermediate support member 250 can include radially spaced-apart openings 260 between longitudinally extending threads. For example, the elongated tubes 270a, 270b and apertures are radially spaced 180 degrees apart. The lead wires 272 can be disposed within the elongated tubes 270 either as bare wire or with an insulation covering. Each elongate tube 270 can terminate proximate the corresponding tracking electrode.

The outer layer 290 can be disposed on the proximal braided member 230, the intermediate support member 250, and the distal braided member 240 and also on the tube 270 as disposed on the outer surface 242 of the distal braided member 240. In some examples, the outer layer 290 can be formed as a coating of reflowable plastic or thermoplastic material that extends over the braided member 224 and seals underlying components of the shaft 202. For instance, the coating can seep by reflowing over braided material of the proximal and distal braided members 230, 240 such as over the threads 342 and into the interstitial spaces 344 of the braided material 340, onto the tubes 270, and onto the threads 342 of the intermediate member 250. In one example, the outer layer 290 is a polyether block amide and, in some examples, is available under the trade designations PEBAX from Arkema, S.A., and VESTAMID E from Evonik Industries, AG.

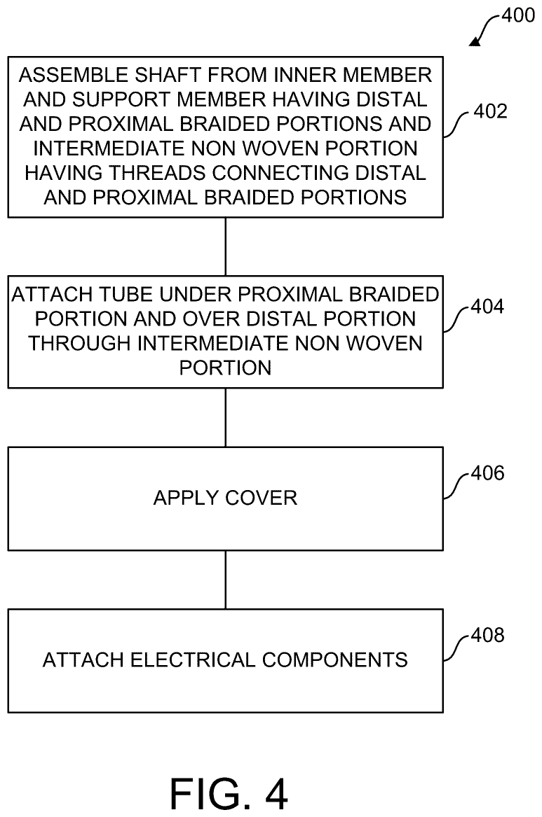

FIG. 4 illustrates an example method 400 of manufacturing a catheter. In one example, method 400 includes a method of constructing a catheter shaft defining a lumen and attached to a tube defining a lead lumen including a conductor lead configured to be attached to a tracking electrode exposed on the catheter shaft. In one example, the elongate support member is formed over an inner member. The support member is formed by weaving threads into a proximal braided member that is longitudinally spaced apart from a distal braided that is also formed by weaving threads. An intermediate member is formed from nonwoven threads connecting the proximal and distal braided members at 402. In one example, an elongated braided member, such as support member 224 is formed by coupling a proximal braided member and a distal braided member to reinforcement member having an opening. The proximal and distal braided members can include an electrically conductive stainless steel braided material. The reinforcement member is a tubular member that, in one example, does not include a braided material. An elongated tube extending longitudinally is attached along the elongated support member.

The tube is disposed longitudinally along the proximal braided member and radially underneath the proximal braided member, through an opening in the threads of the intermediate, nonwoven portion, and then extending along the outer surface of the distal braided member at 404. In the example, the woven proximal and distal braided portions include interstitial spaces that are too small and tightly woven for the tube to pass through. The threads in the nonwoven intermediate portion are spaced apart enough or can be manipulated to be spaced apart enough to pass the tube through such that the tube extends from radially underneath the proximal braided portion to the outer surface of the distal braided portion through the opening in the nonwoven intermediate portion.

The tube and support member receive a cover (or outer layer) at 406. The tube can be connected to the outer surface of the distal braided portion. In one example, a thread, such as a nylon thread, can be longitudinally coiled around the tube and at least the distal braided portion to hold the tube against the outer surface of the distal braided portion. A distal end of the tube is sealed to protect the lead lumen while the outer layer is being applied. The distal end of the tube is sealed to reduce the likelihood of the outer layer filling the lead lumen. Additionally, the distal end of the tube can be reinforced to remain under tension against the distal braided member in preparation for the outer layer. In one example, the distal end of the tube is secured to outer surface of the distal braided member via an attachment member including tape, such as silicone tape, melted extrusions, or a heat shrink tubing applied over the ends and heated. An example of the heat shrink tubing can include PET medical heat shrink tubing. The cover (or outer layer) can be applied via reflow. The attachment member can be removed after the outer layer is applied. A lead conductor is put into a lead lumen of the tube, and the lead conductor is electrically coupled to a tracking electrode disposed on the shaft at 408.

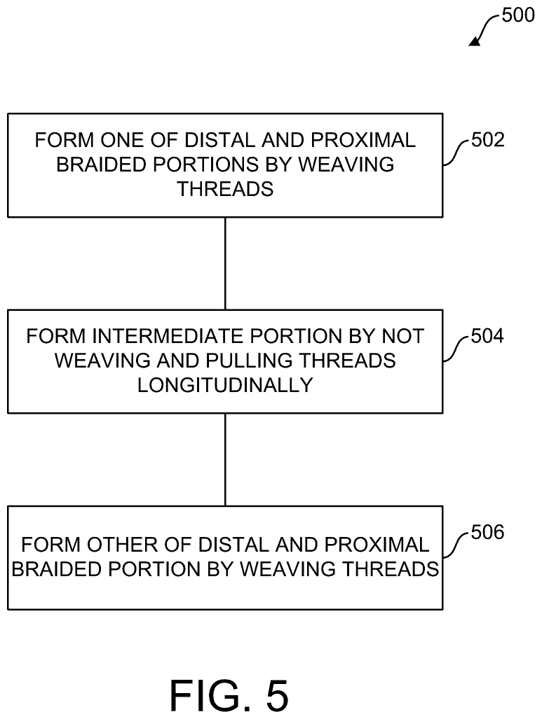

FIG. 5 illustrates an example method 500 of an intermediate member is formed from nonwoven threads connecting the proximal and distal braided members at 402. In the embodiment, a support member is formed by weaving threads along the axis to form braided portions on the inner member, such as with a weaving machine. For instance, the weaving can be formed by weaving along the axis from the proximal end of the inner member to the distal end of the inner member, or vice versa. The weaving machine can be used to form one of the proximal braided portion and the distal braided portion by weaving threads and moving the machine along the axis at 502. At the intermediate portion, the weaving machine pauses weaving but continues to move along the axis at 504. Accordingly, threads from the one of the proximal and distal portions are dragged longitudinally along the axis to form the nonwoven intermediate portion. At the other of the distal braided portion and proximal braided portion, weaving with the weaving machine resumes and forms the other of the distal braided portion and proximal braided portion by weaving threads and moving the machine along the axis at 506.

Various modifications and additions can be made to the exemplary embodiments discussed without departing from the scope of the present disclosure. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A catheter, comprising:
an elongated shaft defining a lumen along a longitudinal axis and having a distal section, the shaft comprising:
a longitudinally extending inner member defining the lumen; and
a support member disposed on the longitudinally extending inner member, the support member including a proximal woven portion and a distal woven portion separated by an intermediate nonwoven portion, the distal woven portion having an outer surface, the proximal woven portion coupled to the distal woven portion by a plurality of longitudinally extending interconnecting threads of the intermediate portion;
an electrode disposed on the distal section of the elongated shaft;
an elongated tube extending longitudinally along the elongated shaft; and
a lead conductor disposed within the tube and electrically coupled to the electrode;
wherein the tube extends longitudinally along the proximal woven portion radially underneath the proximal woven portion, extends longitudinally on the outer surface along the distal woven portion, and extends from underneath the proximal woven portion to the outer surface at the intermediate nonwoven portion through the interconnecting threads.

2. The catheter of claim 1, wherein the proximal woven portion and the distal woven portion includes a stainless-steel braided material.

3. The catheter of claim 2, wherein the braided material includes conductive threads.

4. The catheter of claim 1, wherein the elongated shaft includes an outer layer disposed on the support member.

5. The catheter of claim 4, wherein the outer layer includes a polyether block amide.

6. The catheter of claim 1, wherein the electrode is ring electrode.

7. The catheter of claim 1, wherein the electrode includes a plurality of electrodes, and the tube includes a plurality of tubes.

8. The catheter of claim 1, wherein the shaft includes a proximal end, the proximal end coupled to an electrical connector, and the lead conductor electrically coupled to the electrical conductor.

9. The catheter of claim 1, wherein the proximal braided member includes an inner surface, and the tube extends longitudinally along the inner surface.

10. The catheter of claim 1, wherein the catheter is a guide catheter having a proximal end coupled to a handle.

11. The catheter of claim 1, wherein the proximal woven portion includes a first set of warp and weft threads, the distal woven portion includes a second set of warp and weft threads, and warp threads of the first set extend to the warp threads of the second set as the interconnected threads.

12. The catheter of claim 1, wherein the proximal woven portion and the distal woven portion are characterized by a picks per inch of greater than zero, and the intermediate nonwoven portion is characterized by a picks per inch of zero.

13. The catheter of claim 1, wherein the catheter includes one of a dilator and a guide catheter.

14. The catheter of claim 1, wherein the shaft includes a distal tip section having an ablation electrode assembly.

15. The catheter of claim 1, wherein the tube is attached to the outer surface along the distal woven portion.

16. A system for tracking a catheter during an electrophysiology procedure on a patient, the system comprising:
a catheter disposable within a patient, the catheter comprising:
an elongated shaft defining a lumen along a longitudinal axis and having a distal section, the shaft comprising:
a longitudinally extending inner member defining the lumen; and
a support member disposed on the longitudinally extending inner member, the support member including a proximal woven portion and a distal woven portion separated by an intermediate nonwoven portion, the distal woven portion having an outer surface, the proximal woven portion coupled to the distal woven portion by a plurality of longitudinally extending interconnecting threads of the intermediate portion;
a tracking electrode disposed on the distal section of the elongated shaft;
an elongated tube extending longitudinally along the elongated shaft; and
a lead conductor disposed within the tube and electrically coupled to the electrode;
wherein the tube extends longitudinally along the proximal woven portion radially underneath the proximal woven portion, extends longitudinally on the outer surface along the distal woven portion, and extends from underneath the proximal woven portion to the outer surface at the intermediate nonwoven portion through the interconnecting threads; and
a controller operably coupled to the tracking electrode, the controller configured to receive an electrical signal from the tracking electrode and determine a position of the catheter with respect to the patient.

17. The system of claim 16, wherein the controller if further configured to generate an electroanatomical map of the heart of the patient.

18. The system of claim 16, wherein the controller is configured to determine the position of the catheter via an impedance-based process.

19. A method of manufacturing a catheter, the method comprising:
forming a distal section of an elongate shaft from an inner member defining a lumen, a support member disposed on the inner member, the support member including a proximal woven portion, and a distal woven portion separated by an intermediate nonwoven portion, the distal woven portion having an outer surface, the proximal woven portion coupled to the distal woven portion by a plurality of longitudinally extending interconnecting threads of the intermediate portion;
attaching an elongated tube longitudinally along the elongated shaft, wherein the tube extends longitudinally along the proximal woven portion radially underneath the proximal woven portion, extends longitudinally on the outer surface along the distal woven portion, and extends from underneath the proximal woven portion to the outer surface at the intermediate nonwoven portion through the interconnecting threads;
attaching an electrode on the distal section of the elongated shaft; and
electrically coupling a lead conductor disposed in the tube to the electrode.

20. The method of claim 19, wherein the forming of the distal section includes forming one of the proximal woven portion and the distal woven portion by weaving threads, forming the intermediate portion by longitudinally pulling the threads and not weaving the threads, and forming the other of the proximal woven portion and the distal woven portion by weaving the threads.
